(19) United States
(12) Patent Application Publication
(10) Pub. No.: US 2012/0035510 A1
(43) Pub. Date: Feb. 9, 2012

(54) METHOD FOR EVALUATING THE HOLD ON A LOWER LIMB OF A KNITTED ELASTIC VEIN COMPRESSION ORTHOSIS

(76) Inventors: Francois Cros, Ivry Sur Seine (FR); Aude Lescaille, Lyon (FR)

(21) Appl. No.: 13/201,299

(22) PCT Filed: Feb. 11, 2010
(86) PCT No.: PCT/FR2010/050232

§ 371 Date: Oct. 14, 2011

(30) Foreign Application Priority Data
Feb. 13, 2009 (FR) 09 00653

(51) Int. Cl. A61B 5/103

(52) U.S. Cl. 600/592

(57) ABSTRACT

The orthosis (10) comprises: a foot portion (12); a leg portion (14) exerting a therapeutic compression textile pressure on the leg; and a rib-knitted end portion (16) locally exerting a holding textile pressure for preventing the terminal portion from sliding downwards under the effect of the elastic return force. (F) from the leg portion that is stretched in the longitudinal direction. The method comprises obtaining data representative of: morphological characteristics of the limb; rheological characteristics of the leg portion of the orthosis; friction characteristics at the interface between the limb and the leg portion of the orthosis; rheological characteristics of the end portion of the orthosis; and friction characteristics at the interface between the limb and the end portion of the orthosis. The following step consists in numerically simulating the action on the limb of the leg portion and of the end portion of the orthosis, and in providing at least one indicator that quantifies the way the orthosis will hold on the limb.

The invention relates to elastic venous compression (EVC) orthoses that are indicated for various clinical manifestations of venous insufficiency of the lower limbs.

These orthoses, previously known as “elasticized stockings” or “elasticized socks” are textile medical devices that produce a therapeutic effect by compressing the lower limbs, as contrasted with “support stockings” (or indeed “antifatigue stockings”) and with “fashion stockings” or “fashion socks”, which are not medical devices with a therapeutic intention.

EVC orthoses are designed to produce a therapeutic effect by compressing the lower limb over a greater or lesser extent, with a profile that tapers off going upwards from the ankle. Depending on the type of orthosis, the pressure measured at the ankle may lie in the range 10 millimeters of mercury (mmHg) to more than 36 mmHg (i.e. 13 hectopascals (hPa) to 48 hPa, where mmHg is nevertheless in common use as a pressure measurement unit in the field of phlebology and medical compression). Orthoses are arranged in the ASQUAL reference system in four textile classes, namely class I (13 hPa to 20 hPa≈10 mmHg to 15 mmHg at the ankle), class II (20 hPa to 27 hPa≈15 mmHg to 20 mmHg), class III (27 hPa to 48 hPa≈20 mmHg to 36 mmHg), and class IV (>48 hPa≈>36 mmHg).

EVC orthoses may in particular be in the form of long socks, also known as “half-hose” or “knee socks” (covering the foot, the ankle, and the calf up to just below the knee) with external appearance that is the same as traditional “fashion” socks, but with yarns and knitting selected in such a manner as to obtain effective therapeutic compression, usually class II compression.

The Legger (registered trademark) sock designed and sold by Laboratoires Innothera is an example of such a medical sock forming an EVC orthosis.

An EVC sock of this type essentially comprises a foot portion, a leg portion, and an end portion:
the foot portion, which covers the foot, extends from the toes as far as the ankle bones and covers the instep;
the leg portion, which extends upwards from the ankle to just under the knee, is stretchable in a longitudinal election and in a circumferential direction, and once the orthosis has been put on the limb, it serves to exert a textile compression pressure on the leg at a therapeutic pressure level; and
the end portion, which is generally constituted by a rib-knitted portion, is stretchable in a circumferential direction; once the orthosis has been put on the limb, it serves to exert a holding textile pressure locally at the top of the leg portion at a level suitable for preventing the sock from sliding downwards under the effect of the elastic return force from the leg portion that is stretched in the longitudinal direction.

The starting point of the invention is the observation that patients are often confronted with problems of the orthosis holding up (or “holding”, the terms “holding” and “holding up” both being used in the present description), i.e. once the orthosis has been put into place, it sometimes suffers from the drawback of sliding down the leg under the effect of the elastic return force from the portion that has been stretched.

This phenomenon depends on numerous factors, not only factors specific to the orthosis (e.g. the fact that the rib-knitted end portion provides tightness to a greater or lesser extent) but also, and above all, on factors that are extrinsic, depending on the patient, on whether the orthosis is put on as a good or a poor fit, etc.

Various techniques do indeed exist for evaluating or modeling the pressure profile exerted by an EVC orthosis and its effects on the venous network, as described for example in WO 2006/087442 A or FR 2 882 172 A (Laboratoires Innothéra) or by Rong et al. in Objective evaluation of skin pressure distribution of graduated elastic compression stockings, Dermatol. Surg. 2005; 31: pp 615-624 (2005).

However, those studies were performed using, a priori, under the ideal assumption of an orthosis being properly held up and properly fitted, and they do not give any indication suitable for quantifying defective holding up and/or defective fitting.

Unfortunately, an orthosis that does not hold up properly on a limb does not give the desired effects in terms of venous return, i.e. because it is wrongly positioned it does not give the results that the known modeling techniques serve to evaluate.

One of the objects of the invention is specifically to remedy that problem, by making available to EVC orthosis manufacturers and researchers a method that makes it possible:
to quantify the various factors contributing to the phenomenon whereby, once in place, the orthosis can sometimes slide down on the leg under the effect of the elastic return force from the portion that was stretched; and
to model this phenomenon so as to be able to evaluate how well an orthosis holds up on the leg in a very wide variety of situations and for different orthoses.

Such a study implies in particular taking account of factors that are not taken into account in the above-mentioned known modeling techniques. This applies in particular to the coefficient of friction between the limb and the “rib-knitted” end portion, in order to prevent the sock from sliding downwards under the effect of the elastic return force from the leg portion that has been stretched in the longitudinal direction: it has been found that it is perfectly possible for two socks to present the same pressure profile and to hold up well on the leg, but to have two very different coefficients of friction; similarly, two socks presenting two coefficients of friction that are very different, in association with the same pressure profile will have the same effect on venous return, but the way in which they hold up need not be quantified in the same manner.

On the basis of the evaluation performed by the method of the invention, it is potentially possible for the manufacturer to improve the orthosis so as to guarantee that it wears well, while nevertheless seeking the lowest possible pressure at its end portion, i.e. its rib-knitted end.

In order to encourage compliance by the patient, it is important to avoid excessive pressure leading to discomfort or to difficulty in putting the sock on. Furthermore, excessive levels of pressure at the rib-knitted end can lead to undesirable effects such as constriction or occlusion of superficial veins, phenomena that must naturally be avoided.

Finally, understanding the phenomenon makes it possible to set out a certain number of recommendations in selecting orthoses from a pre-existing grid of sizes, so as to prescribe the size that is best suited as a function not only of the patient's morphology, but also of the need for the orthosis to hold up in satisfactory manner on the leg. These recommendations may also lead to emphasizing the importance of ensuring that the orthosis is properly fitted while it is being put on, in so far as the quality of its fitting has an effect not only on the effectiveness of the compression (in the leg portion) but also on the ability of the orthosis to hold up (via its end portion).

The method of the invention is characterized by the following steps:
obtaining first data representative of the morphological characteristics of the limb;
obtaining second data representative of the rheological characteristics of the leg portion of the orthosis;
obtaining third data representative of the friction characteristics at the interface between the limb and the leg portion of the orthosis;
obtaining fourth data representative of the rheological characteristics of the end portion of the orthosis;
obtaining fifth data representative of the friction characteristics at the interface between the limb and the end portion of the orthosis;
obtaining sixth data representative of the positioning of the leg portion of the orthosis on the patient's limb, said sixth data including a positioning height (h) for the end portion on the limb, and a quality of fitting (BMP, MMP) of the leg portion; and
from said data, numerically simulating the action of the leg portion and of the end portion of the orthosis on the limb, and delivering at least one indicator quantifying how the orthosis will hold on the limb.

This method is most advantageously applicable to the problem of EVC socks holding up on a leg, i.e. to orthoses of the “long sock” or “half-hose” type of the so-called “AD” format, i.e. in which the end portion comes up to just beneath the knee once the orthosis has been put on.

Nevertheless, the method of the invention is equally applicable to evaluating orthoses of the “thigh-hose” type (“GH” format) going up to the top of the thigh.

According to various advantageous subsidiary characteristics of the invention:
the numerical simulation means include means suitable for using said first, second, and sixth data to evaluate the elastic return force exerted by the leg portion that is stretched in the longitudinal direction;
the numerical simulation means include means suitable for using said first, fourth, and sixth data to evaluate said holding textile pressure exerted by the end portion that is stretched in the circumferential direction;
the numerical simulation means are also suitable for using said first, second, third, and sixth data to evaluate the friction force exerted at contact between the limb and said leg portion that is stretched in the longitudinal direction;
the numerical simulation means are also suitable for using said first, third, and sixth data, said elastic return force, and said friction force, to evaluate the theoretical textile pressure that needs to be exerted in order to hold the orthosis at its initial positioning height; and
the indicator quantifying the ability of the orthosis to hold up on the limb includes: a characteristic giving the variations in the textile holding pressure as a function of said data; an indicator quantifying the sensitivity of the orthosis to its positioning height and/or to the quality of its fitting; and/or an indicator of the holding fraction of the orthosis for a set of configurations corresponding to different positioning heights and/or qualities of fitting.

There follows a description of an embodiment of the device of the invention given with reference to the accompanying drawings in which the same numerical references are used from one figure to another to designate elements that are identical or functionally similar.

FIG. 1 is a general view of an orthosis of the invention, in the free state.

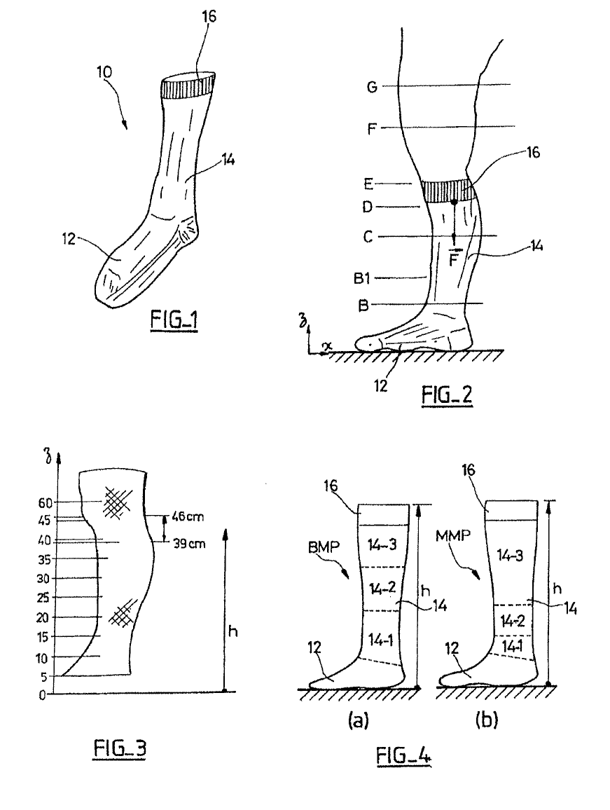

FIG. 2 is an elevation of the same orthosis, in place on a limb.

FIG. 3 is an elevation view showing the possible variations in the height for the position of the end portion on the limb.

FIGS. 4a and 4b are diagrams respectively showing how an orthosis may be fitted well or poorly on the limb.

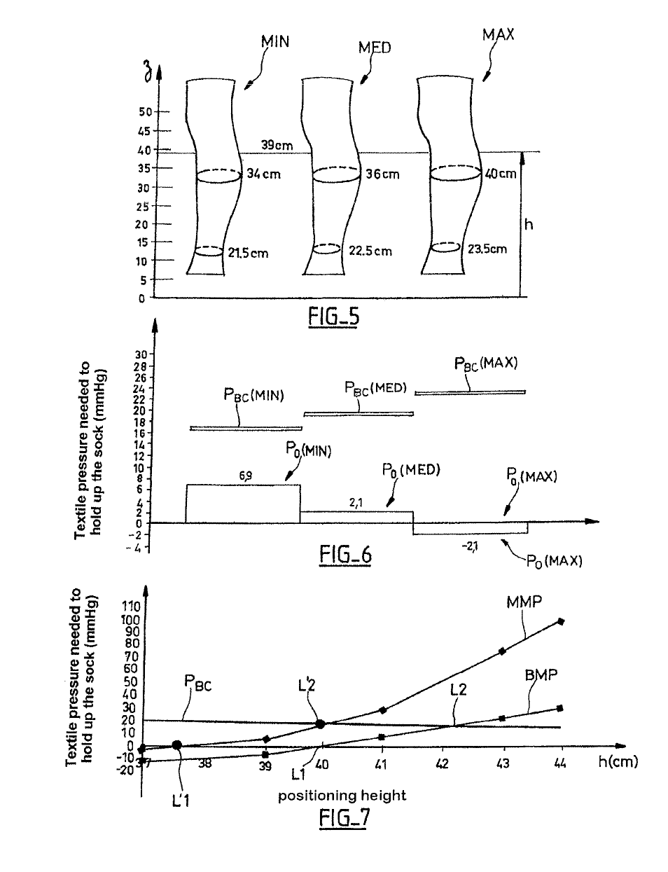

FIG. 5 is a diagram showing the incidence of the morphology of the limb on various formats (minimum, middle, and maximum) in the grid of sizes for a given orthosis.

FIG. 6 shows the textile pressure values needed for holding up a given orthosis for the various dimensions shown in FIG. 5.

FIG. 7 shows the textile pressure variations needed for holding up the orthosis as a function of the positioning height and depending on whether it has been fitted well or poorly by the patient.

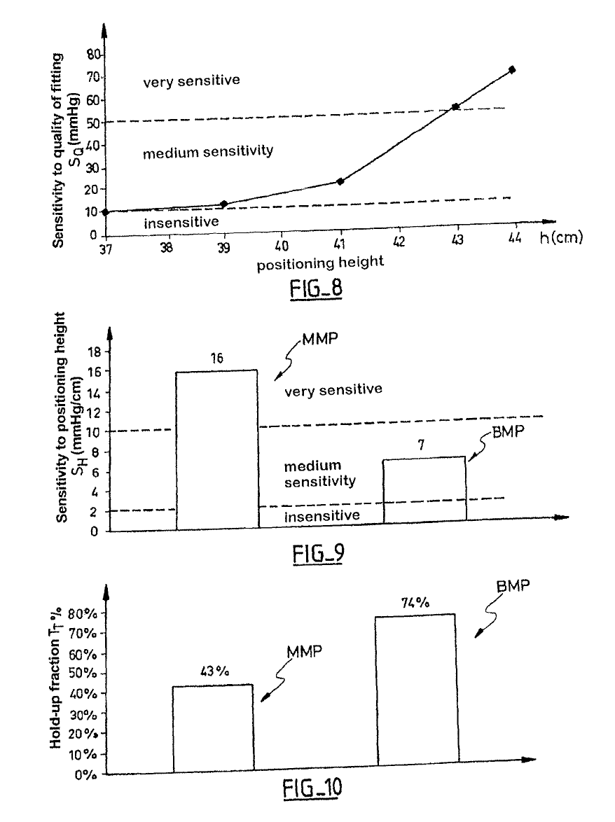

FIG. 8 shows variations in the index representing sensitivity to the quality with which the orthosis is fitted, as a function of positioning height.

FIG. 9 is a histogram showing sensitivity to positioning height in various configurations, when the orthosis is fitted well or poorly.

FIG. 10 is a histogram showing the holding fraction in various configurations for the orthosis being fitted well or poorly.

In FIG. 1, reference 10 is a general reference for an EVC sock that comprises:
a foot portion 12 extending from the toes to the ankles and covering the instep;
a leg portion 14 that is stretchable in the longitudinal direction (i.e. in the direction of the vertical axis z shown in FIG. 2) and in the circumferential direction (“radially” stretchable). This leg portion 14 extends from the ankle region, covering the ankle and the calf up to a level situated below the knee; and
an end portion 16 that is stretchable mainly in the circumferential direction, typically a portion that is rib-knitted. The role of this end portion 16 is to prevent the leg portion sliding downwards under the effect of its elastic return force, and of the various movements and stresses to which the sock is subjected while it is being worn by the patient, which return force is the result of the leg portion being stretched in the longitudinal direction.

In order to enable the lower limbs to be strongly compressed, the leg portion 14 is made using knitting of texture that is tight to a greater or lesser extent, having an elastic weft yarn incorporated therein, generally comprising covered spandex.

More precisely, after being fitted on the limb, the stretched textile of the orthosis exerts compression in the leg portion 14 that results from the return force of the elastic fibers making up the material, and the application of these elastic return forces on the perimeter of the outline gives rise, at any given point, and in application of Laplace's law, to local pressure that is inversely proportional to the radius of curvature of the outline at that point.

This pressure is the “textile pressure” as defined and calculated in accordance with French standard NF G 30-102b. In the present description, the term “pressure” is used to designate the mean of the normalized compression pressures exerted locally at a given height along an outline (circular or elliptical outline in the approximation of a model leg).

The knit and the yarns, and also the size of the rows of knitting, are selected so as to apply predetermined pressures at different heights up the leg, e.g. at the height of the ankle, at the start of the calf, at the calf, etc. These various pressures are defined for each compression class by reference to metrological templates such as the model leg of French standard NF G 30-102b, Appendix B, or the model leg of the Hohenstein type as specified in German reference RAL-GZ 387. The various corresponding heights, shown in FIG. 2, are written B, B1, C, . . . , by convention.

Because of the stretching of the leg portion 14 once it is in place on the limb, and because of various stresses such as rubbing, slipping, etc., this leg portion on its own would generally tend to slide down the leg, thereby losing the desired therapeutic effect of compression in that region.

In order to prevent that phenomenon, the rib-knitted end 16 is designed to exert locally a holding textile pressure that is sufficient to counter the force F tending to urge the proximal end of the leg portion downwards (where F is the resultant of all of the stresses tending to cause this leg portion 14 to slide down the limb).

Until now, the circumferential extensibility (i.e. extensibility in the radial direction) of the rib-knitted end portion 16 has been determined more or less empirically by experience, seeking to find a compromise between:
a low holding textile pressure that is comfortable for the wearer and thus encourages compliance by the patient, but that, in numerous situations, does not enable the sock to be held in place in satisfactory manner; and
a strong holding textile pressure that serves under all circumstances to ensure that the sock holds up well on the leg, but that is uncomfortable for the patient and can even in certain circumstances give rise to a phenomenon of veins being constricted or occluded at the location of the end portion.

The approach of the invention consists in modeling this phenomenon so as to make it possible:
to analyze the behavior of existing orthoses in the various configurations that are likely to be encountered for a given orthosis;
to establish recommendations, in particular concerning the quality with which the sock is put into place and its positioning height; and
to provide potential improvements to existing orthoses, by optimizing the holding textile pressure exerted by the rib-knitted end portion.

Evaluating the force F exerted by the leg portion 14 makes it possible to quantify the level of tightness, i.e. the holding textile pressure, that needs to be exerted by the end portion 16 of the orthosis in order to guarantee that the leg portion 14 is held in place, thus enabling the compression needed for obtaining the looked-for therapeutic effect to be applied in satisfactory manner to the leg.

The action of the leg portion 14 and of the end portion 16 of the orthosis on the limb can be simulated by calculating mechanical lengthening and friction in two dimensions (the longitudinal direction and the radial direction), with integration up the height and around the circumference.

The input parameters are as follows:

1) Elasticity of the orthosis: these are intrinsic rheological characteristics associated with the way the weft yarns are knitted and the stitches selected for the leg portion 14 and for the end portion 16. These rheological characteristics, i.e. the relationship giving the applied tension T as a function of the deformation e, may be determined by conventional dynamometric measurements, or indeed by using an extensometer such as that described in WO 01/11337 A1 (Innothera Topic International). On the basis of these measurements, it is possible to extrapolate a relationship that makes it possible to determine tension at any point of the orthosis as a function of deformation, both in the height direction (longitudinal elasticity) and in the radial direction (elasticity in the circumferential direction).

2) Friction coefficient: this coefficient characterizes the interface between the skin and the leg portion or the end portion. This coefficient depends on the materials selected for each of these portions (e.g. the presence of cotton increases friction, and therefore provides an orthosis that has less tendency to slide down than it would have if it is were made entirely out of synthetic material), and also on the characteristics of the wearer's skin: hair distribution, skin dryness, etc. This coefficient of friction is determined experimentally.

3) Morphology: the morphology of the wearer has an influence mainly on the radial tension of the orthosis:

at a given height, a thicker leg presents a longer circumference and, for a given orthosis, gives rise to a higher textile pressure. In the description below, consideration is given to series of morphologies corresponding to predetermined size grids of an orthosis as established relative to model legs of a reference limb. It is also possible to take into consideration the real morphology of a limb of a patient or of a population of patients, in particular by taking measurements by laser plethysmography, e.g. by means of an installation such as that described in FR 2 774 276 A1 and FR 2 804 595 A1 (Innothera Topic International), making it possible to draw up a very accurate map of an individual's limb along successive sections of the limb. By combining this morphological data with the rheological data specific to the orthosis, it is possible to calculate the pressure exerted by the orthosis at any point on the leg. The calculation may in particular apply a technique such as that described in WO 2004/095342 A2 (Laboratoires Innothera), that explains how these two data series can be combined so as to produce a complete map of pressures applied to the limb.

4) Positioning of the orthosis: this parameter, which mainly influences the longitudinal section of the orthosis, is made up of two subparameters, namely:

a) positioning height, i.e. the height of the proximal end of the end portion 16, in other words the height up to which the orthosis extends once it has been put on by the patient. FIG. 3 illustrates this parameter: for an orthosis having a nominal positioning height (just below the popliteal fossa) that is for example 39 centimeters (cm), if the patient exerts too much traction while putting the sock on, then the real positioning height h may go beyond said nominal value, e.g. over a range 39 cm to 46 cm, which can have a major effect on how well the orthosis holds up: if the sock is stretched too far upwards, then the elastic return force F is greater and the sock will have a much greater tendency to slide down;

b) quality of fitting: unlike everyday socks, an EVC sock needs to be put on in compliance with precise recommendations, with the sock being fitted progressively and massaged into place while it is being put on. FIG. 4a shows such a good fit situation (BMP) in which the bottom zone 14-1, middle zone 14-2, and top zone 14-3 of the leg portion 14 are uniformly distributed along the leg. In contrast, if the patient is content to get the sock past the ankle and then merely to pull up its end portion, then the top zone 14-3 of the leg portion will be stretched much more than the other two zones 14-1 and 14-2, as shown in FIG. 4b which shows such a poor fit situation (MMP). The large amount of stretching of the top portion 14-3 tends considerably to increase the elastic return force F exerted on the end portion 16, thereby contributing to causing it to slide downwards. In other words, for the same positioning height, a sock that is poorly fitted will have much more difficulty in staying in position than it would if it had been fitted in compliance with the manufacturer's recommendations.

There follows an explanation of how these various parameters can be combined with one another in order to evaluate the sensitivity of an orthosis to how it is positioned (positioning height and fitting quality) and also to the morphology of the limb, where these factors are determining factors concerning an orthosis that holds up effectively.

The explanation begins with reference to FIGS. 5 and 6 and concerns the effect of the morphology of the limb on the way the orthosis holds up.

FIG. 5 shows three virtual leg models corresponding respectively to the minimum (MIN), the middle (MED), and the maximum (MAX) of the size grid for a given orthosis, the orthosis being put on in all three circumstances in such a manner as to be positioned at the same height h (h=39 cm in the example shown), and with fitting of good quality (BMP) as defined above.

These standard sizes are established with reference to standardized model legs, but it would equally be possible to study sensitivity to morphology on the basis of measurements actually made on real legs of various patients, e.g. mapped by means of a laser plethysmography device, as mentioned above.

For the three morphologies MIN, MED, and MAX taken into consideration, it can be seen that the minimum circumference of the leg at the ankle lies typically in the range 21.5 cm to 23.5 cm (circumference at the height B of FIG. 2), whereas its maximum circumference, at the calf, lies typically in the range 34 cm to 40 cm (circumference at the height C in FIG. 2).

On the basis of this data, and using the rheological data, it is possible to calculate firstly the pressure P_{BC} actually exerted by the end portion 16 on its own (the rib-knitted portion), and secondly the minimum holding pressure P₀ that needs to be exerted in order to counter the return force F (FIG. 2) exerted by the leg portion as a result of the elastic return due to this portion being stretched once it has been put on the limb.

These values P_{BC} and P₀ as calculated for the three different morphologies are marked on FIG. 6.

It can be seen that for the maximum MAX of the size grid, the pressure P₀ has a negative value, which means that the compression exerted on the leg by the leg portion of the orthosis itself delivers sufficient holding power to ensure that the orthosis stays in place without it being necessary to add pressure at its end.

In other words, for this morphology, the orthosis will hold up in satisfactory manner even in the absence of a rib-knitted end portion.

In any event, in all of these configurations, the pressure P_{BC} exerted by the rib knitting is always greater than the pressure P₀ needed to hold up the orthosis. Providing the nominal positioning height and good fitting are complied with, this means that the presence of the rib-knitted end portion guarantees that the orthosis will hold up under all circumstances, including a safety margin (P_{BC}-P₀) of sufficient size.

Another important factor to be taken into consideration when evaluating how well the orthosis holds up is how it is positioned, as explained below with reference to FIGS. 6 and 7.

As mentioned above, the positioning parameter may be considered as two subparameters, namely: i) the positioning height h; and ii) the quality of fitting, which may be good (BMP) or poor (MMP).

In order to study the impact of these two parameters, calculation grids have been defined, one complying with fitting of good quality BMP and the other stimulating fitting of poor quality MMP. Furthermore, for each of the two series, different positioning heights h have been simulated: for example, for a nominal positioning h=39 cm, measurements have been taken for h=37 cm, 38 cm (sock not pulled up far enough), and h=40 cm, 41 cm, 42 cm, 43 cm, and 44 cm (sock pulled up too far, which corresponds to a situation that is relatively frequent).

These two series of measurements have made it possible to establish two textile pressure characteristics as a function of positioning height, and labeled MMP and BMP in FIG. 7. This figure also shows the textile pressure P_{BC} exerted by the rib-knitted end of the top portion, which value thus corresponds to the theoretical limit for holding up the orthosis.

An examination of FIG. 7 shows that the holding limit for the leg portion on its own, considered independently of the rib-knitted end, corresponds to a positioning height h=40 cm (point L1) for good fitting, which value is greatly reduced, going down to 37.5 cm (point L′1) with poor fitting.

In the same way, the holding limit using a rib-knitted end portion goes down from h=42 cm (point L2) when associated with good fitting to h=40 cm (point L′2) when associated with poor fitting, thereby demonstrating the considerable incidence of this parameter on the orthosis holding up.

From the above analysis, it is possible to define various criteria enabling the holding up of the orthosis to be quantified simply.

These criteria, shown with reference to FIGS. 8, 9, and 10 respectively are as follows:
the sensitivity S_{Q} to the quality of fitting;
the sensitivity S_{H} to the positioning height; and
the holding fraction T_{T}.

The criterion S_{Q} quantifies the textile pressure difference for the rib-knitted end portion that is needed to hold up the orthosis, this difference being considered between the good fitting situation BMP and the poor fitting situation MMP. This criterion, expressed in mmHg, as a function of the positioning height h, is represented by the curve in FIG. 8. As a function of the value of S_{Q}, three levels of sensitivity may be defined:
S_{Q}<10 mmHg: low sensitivity to quality of fitting;
S_{Q} lying in the range 10 mmHg to 50 mmHg: medium sensitivity to quality of fitting; and
S_{Q}>50 mmHg: high sensitivity to quality of fitting.

The criterion S_{H} that serves to quantify the sensitivity to the positioning height is calculated from the spatial derivative of the textile pressure. The result is expressed in mmHg per centimeter (mmHg/cm), and it represents the textile pressure difference needed to ensure the orthosis holds up between two heights that are spaced apart by 1 cm. The calculation is performed for two quality of fitting configurations, good fitting BMP and poor fitting MMP. The results obtained over a set of positioning heights tested on the same subject are summarized by the histograms of FIG. 9. Depending on the value of S_{H}, it is possible to define three sensitivity levels:
S_{H}>2 mmHg/cm: little sensitivity to positioning height;
S_{H} lying in the range 2 mmHg/cm to 10 mmHg/cm: medium sensitivity to positioning height; and
S_{H}>10 mmHg/cm: great sensitivity to positioning height.

Finally, the holding fraction T_{T} corresponds to the percentage represented by the range of positioning heights for which the orthosis is held in place with the help of the rib-knitted end portion, compared with the range of positioning heights tested. This fraction is calculated as follows:

T_{T}=(effective holding limit−minimum positioning height)÷(maximum positioning height−minimum positioning height)

The results obtained are summarized by the histograms of FIG. 10, respectively for poor quality fitting MMP and for good quality fitting BMP: specifically, this means that with poor fitting, the orthosis can theoretically be in a satisfactory holding condition for only 43% of the positioning heights tested, whereas with good fitting it can be under such conditions in 74% of heights.

Analyzing these quantitative criteria for such and such a category of orthosis makes it possible to determine whether each criterion presents little/medium/great sensitivity to positioning, revealing whether the positioning sensitivity is the result of specific sensitivity to positioning height and/or to the quality of fitting.

For a given orthosis, it is thus possible to establish recommendations such as:
decreasing, retaining, or increasing the textile pressure exerted by the rib-knitted end portion;
in the instructions and at the time of prescription, emphasizing mainly the importance of good quality fitting and/or of complying with the positioning height (and in particular on the need to avoid pulling the orthosis up too high); and
optionally modifying such and such an orthosis reference to a greater extent in order to improve its performance in terms of holding up on a leg, for a broader population of patients and for a wider variety of positioning conditions (positioning height and quality of fitting).

1. A method of evaluating the hold of a knitted elastic venous compression orthosis (10) on the lower limb of a patient, the orthosis comprising:
a foot portion (12);
a leg portion (14) extending upwards from the ankle and stretchable in a longitudinal direction and in a circumferential direction, the leg portion being suitable, once the orthosis has been put on the limb, for exerting a compression textile pressure thereon at a therapeutic pressure level; and
an end portion (16) that is stretchable in a circumferential direction, in particular a rib-knitted end portion, the end portion being suitable, once the orthosis has been put on the limb, for exerting locally thereon a holding textile pressure at a level that is suitable for preventing the end portion sliding downwards under the effect of the elastic return (F) from the leg portion that is stretched in the longitudinal direction;
the method being characterized by the following steps:
obtaining first data representative of the morphological characteristics of the limb;
obtaining second data representative of the rheological characteristics of the leg portion of the orthosis;
obtaining third data representative of the friction characteristics at the interface between the limb and the leg portion of the orthosis;
obtaining fourth data representative of the rheological characteristics of the end portion of the orthosis;
obtaining fifth data representative of the friction characteristics at the interface between the limb and the end portion of the orthosis;
obtaining sixth data representative of the positioning of the leg portion of the orthosis on the patient's limb, said sixth data including a positioning height (h) for the end portion on the limb, and a quality of fitting (BMP, MMP) of the leg portion; and
from said data, numerically simulating the action of the leg portion and of the end portion of the orthosis on the limb, and delivering at least one indicator quantifying how the orthosis will hold on the limb.

2. The method of claim 1, wherein the numerical simulation step comprises a step of using said first, second, and sixth data to evaluate the elastic return force (F) exerted by the leg portion (14) that is stretched in the longitudinal direction (z).

3. The method of claim 1, wherein the numerical simulation step includes a step of using said first, fourth, and sixth data to evaluate said holding textile pressure exerted by the end portion (16) that is stretched in the circumferential direction.

4. The method of claim 1, wherein the numerical simulation step includes a step of using said first, second, third, and sixth data to evaluate the friction force exerted at contact between the limb and the leg portion (14) that is stretched in the longitudinal direction (z).

5. The method of claim 4, wherein the numerical simulation step includes a step of using said first, fifth, and sixth data, said elastic return force, and said friction force to evaluate the theoretical textile pressure to be exerted in order to hold the orthosis at its initial positioning height.

6. The method of claim 1, wherein said indicator quantifying the way the orthosis is held on the limb comprises a characteristic (S_{Q}) giving variations of said holding textile pressure as a function of said data.

7. The method of claim 1, wherein said indicator quantifying the holding of the orthosis on the limb comprises an indicator (S_{H}) quantifying the sensitivity of the orthosis to the positioning height and/or to the quality of fitting.

8. The method of claim 1, wherein said indicator quantifying the hold of the orthosis on the limb comprises an indicator (T_{T}) of the hold fraction of the orthosis for a set of configurations corresponding to different positioning heights and/or to the qualities of fitting.
